(19) United States
(12) Patent Application Publication
(10) Pub. No.: US 2013/0211144 A1
(43) Pub. Date: Aug. 15, 2013

(54) MULTI-STAGE REACTION FOR MITIGATINGTHE PRESENCE OF UNWANTED CATIONS

(71) Applicant: EE-TERRABON BIOFUELS, LLC., (US)

(72) Inventors: Cesar B. GRANDA, College Station, TX (US); Michael Kyle Ross, Bryan, TX (US); Jubo Zhang, College Station, TX (US); Gary W. Luce, Spring, TX (US); John A. Spencer, Cypress, TX (US); Simon H. Upfill-Brown, Houston, TX (US); John Tjaden, Pearland, TX (US); Michael Landoll, Lake Jackson, TX (US)

(73) Assignee: EE-TERRABON BIOFUELS, LLC., New Braunfels, TX (US)

(21) Appl. No.: 13/762,917

(22) Filed: Feb. 8, 2013

Related U.S. Application Data
(60) Provisional application No. 61/597,629, filed on Feb. 10, 2012.

(51) Int. Cl. C07C 45/54

(52) U.S. Cl. CPC C07C 45/54
USPC 568/397

(57) ABSTRACT

Methods for producing organic products that may include the steps of providing a feed stream comprising a first organic salt and a second organic salt to a reactor; reacting the feed stream at a first temperature to convert at least some of the first organic salt to the organic products, wherein reacting the feed stream results in a first product stream comprising the organic products and second organic salt; separating at least a portion of the organic products from the first product stream resulting in a first reduced product stream comprising second organic salt; and reacting the first reduced product stream at a second temperature to convert the second organic salt to a second organic products.

### CROSS-REFERENCE TO RELATED APPLICATIONS

This application claims the benefit under 35 U.S.C. §119(e) of U.S. Provisional Patent Application No. 61/597,629 filed Feb. 10, 2012, the disclosure of which is hereby incorporated herein by reference for all purposes.

### STATEMENT REGARDING FEDERALLY SPONSORED RESEARCH OR DEVELOPMENT

Not applicable.

### BACKGROUND

1. Field of the Disclosure

This disclosure generally relates to methods and processes for producing ketones. More specifically, the disclosure relates to methods for producing ketones while mitigating the presence of unwanted feed stream components, such as certain cations.

2. Background of the Disclosure

Converting organic salts, such as carboxylate salts, into other products can occur by thermal decomposition. Such processes are well known and some of them are very old. For instance, as early as 1732, researchers knew of decomposition of potassium acetate, and knew that the liquid generated was not an alcohol. During World War I, large quantities of acetone were made for the war effort by the thermal decomposition of calcium acetate.

It has been observed that for high yields to be obtained, the quick removal of the resulting vapors, such as ketones, are necessary to avoid the degradation of the end product. Literature and research suggest the metal or cation of the salt has a significant effect in the kinetics and yields as well as the temperature needed for the reaction to occur. Different cations require different temperatures for complete conversion and also lower temperatures also minimize the further thermal degradation of the end product.

High temperatures during the ketonization process tend to produce more degradation product by promoting radical formation, which causes polymerization and thus tar formation, and by cracking which results in small gaseous compounds such as methane, ethane and carbon dioxide. Minimizing temperature or the time at which the reaction occurs at high temperatures is, therefore, imperative to improve yields and better quality products.

In the past, only one-stage reactor system was used, and the process was forced to operate at one temperature, which would basically be the lowest temperature required to achieve high conversion of the salts entering the system. Due to the presence of the unwanted cations, even in small quantities, the temperature had to be raised quite considerably generating lower yields.

There are needs in the art for novel methods for producing ketones with higher yields and reduced degradation. There is a great need for reducing or mitigating the presence of unwanted metals or cations present in ketonization feed streams.

### SUMMARY

Embodiments disclosed herein pertain to a method for producing organic products that may include providing a feed stream comprising a first organic salt and a second organic salt to a reactor; reacting the feed stream at a first temperature to convert at least some of the first organic salt to the organic products, wherein reacting the feed stream results in a first product stream comprising the organic products and second organic salt; separating at least a portion of the organic products from the first product stream resulting in a first reduced product stream comprising second organic salt; and reacting the first reduced product stream at a second temperature to convert the second organic salt to a second organic products.

In aspects, the first temperature may be different than the second temperature. The reacting the feed stream may occur in the reactor. The reacting the first reduced product stream may occur in a second reactor. The first organic salt may include magnesium or calcium. The feed stream may be resultant from acidogenic fermentation or the alkali treatment of a bioproduct. The portion of the organic products and a portion of the second organic products may be gaseous. The first organic salt may include an alkali metal.

The first temperature may be less than the second temperature. Reacting the feed stream may occur in the reactor. Reacting the first reduced product stream may occur in a second reactor. The first organic salt may be magnesium organic salt. Reacting the feed stream may occur in the reactor, and reacting the first reduced product stream may occur in a second reactor.

Reacting the feed stream may occur in the reactor, and reacting the first reduced product stream may occur in a second reactor. The first temperature may be less than the second temperature, and reacting the feed stream and reacting the first reduced product stream may occur in the reactor.

In aspects, reacting the feed stream and reacting the first reduced product stream may occur in the reactor. The first organic salt may be produced from a magnesium carbonate or magnesium hydroxide buffered fermentation reaction.

Other embodiments of the disclosure pertain to a method for improving a pyrolysis reaction that may include providing a feed stream comprising organic salt of certain cations to a reactor; reacting the feed stream at a first temperature to convert at least some of the organic salt of certain cations to a first organic products, further whereby reacting the feed stream results in a first product stream comprising first organic products and unconverted organic salt of certain cations; separating at least a portion of the first organic products from the first product stream resulting in a first reduced product stream comprising unconverted organic salt cations; and reacting the first reduced product stream at a second temperature to convert at least some of unconverted organic salt cations to a second organic products.

In aspects, the first temperature may be different to the second temperature, reacting the feed stream occurs in the reactor, and reacting the first reduced product stream occurs in a second reactor.

The feed stream may be resultant from acidogenic fermentation of a bioproduct selected from the group consisting of agricultural crops and biodegradable wastes. In embodiments, reacting the feed stream further may result in an ash, and at least some of the ash may be recycled and used as a buffering agent in the acidogenic fermentation to control pH.

The first temperature may be in the range of about 340° C. to 450° C., wherein the reacting the feed stream may be by thermal decomposition. At least some of the cations of the carboxylate organic salt may include an alkali metal.

The first temperature may be less than the second temperature, reacting the feed stream may occur in the reactor, and reacting the first reduced product stream may occur in a second reactor. Reacting the feed stream may occur in the reactor, and reacting the first reduced product stream may occur in a second reactor.

In aspects, the first temperature may be less than the second temperature, and wherein reacting the feed stream and reacting the first reduced product stream may occur in the reactor. The feed stream may be resultant from acidogenic fermentation or the alkali treatment of a bioproduct selected from the group consisting of agricultural crops and biodegradable wastes.

These and other embodiments, features and advantages will be apparent in the following detailed description and drawings.

### BRIEF DESCRIPTION OF THE DRAWINGS

For a more detailed description of the present invention, reference will now be made to the accompanying drawings, wherein:

FIG. 1 shows TGA curves of carboxylate salts with different cations.

FIG. 2 shows a process flow diagram of a two-stage ketonization reactor system with multiple reactors, according to embodiments of the disclosure.

FIG. 3 shows a process flow diagram of a two-stage ketonization reactor system within the same reactor, according to embodiments of the disclosure.

### DETAILED DESCRIPTION

Herein disclosed are novel methods and processes that pertain to producing ketones and mitigating the effects of certain undesired cations during ketonization. Such methods may include the steps of providing a feed stream comprising a first organic salt and a second organic salt to a reactor; reacting the feed stream at a first temperature to convert at least some of the first organic salt to the ketone, further wherein reacting the feed stream results in a first product stream comprising the ketone and second organic salt; separating at least a portion of the ketone from the first product stream resulting in a first reduced product stream comprising second organic salt; and reacting the first reduced product stream at a second temperature to convert the second organic salt to a second ketone.

Other methods disclosed herein may be used for improving a ketonization reaction. Such methods may include the steps of providing a feed stream comprising organic salt of certain cations to a reactor; reacting the feed stream at a first temperature to convert at least some of the organic salt of certain cations to a first ketone, further whereby reacting the feed stream results in a first product stream comprising first ketone and unconverted organic salt of certain cations; separating at least a portion of the first ketone from the first product stream resulting in a first reduced product stream comprising unconverted organic salt of certain cations; and reacting the first reduced product stream at a second temperature to convert at least some of unconverted carboxylate salt cations to a second ketone.

Embodiments described herein may pertain to by way of example and illustration two stages; however, any number of stages with varying temperature may be employed to improve the efficiency of the ketonization process.

Organic salts may be, for example, carboxylate salts.

In accordance with embodiments described herein, it may be understood that smaller cations and divalent cations allow ketonization reactions to be performed at lower temperatures and allow for higher yields to occur. This effect seems to be two-edged in the sense the data and evidence shows higher yields are obtained not only because of the lower temperatures at which the reaction is performed, but also due to an intrinsic effect of the divalent cation in the reaction. For example, calcium salts react at about the same temperature as sodium salts, however, higher yields are obtained from the calcium salts rather than from sodium. In this manner, it is preferable to move up and towards the di-valent cations group in the first two groups or columns of the alkali metals in the periodic table.

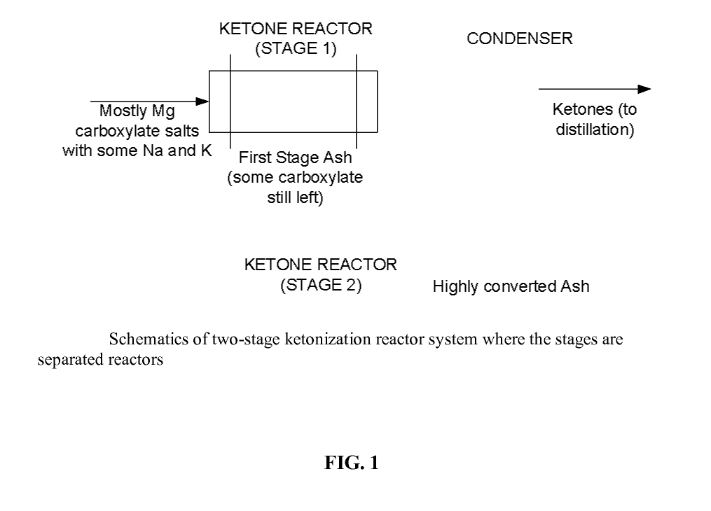

Results showing smaller cations and divalent cations react at a lower temperature are clearly seen on the TGA curves of the carboxylate salts of different cations of FIG. 1. Conceivably, the most adequate cation would be beryllium, as it is the smallest of the di-valent cations; however, the toxicity of beryllium precludes its use efficiently in an industrial setting. Therefore, magnesium may be a suitable alternative to be the cation of choice, with ketonization occurring possible at relatively low temperatures (e.g., about 350° C.).

In overall production, carboxylate salts may be generated industrially from the acidogenic fermentation of biodegradable materials, such as energy crops and biodegradable wastes. This fermentation produces carboxylic acids which may range from acetic acid (C2) all the way to octanoic acid (C8). In view of what has been mentioned above, the buffering agent of choice to control pH as the acids are formed in this fermentation would be magnesium carbonate and/or magnesium hydroxide. The fermentation product would be, in theory, the magnesium salts of the carboxylic acids, which can then be purified and dewatered and fed to the thermal conversion ketonization reactor, where the corresponding ketones of the acids would be generated as vapors, removed and condensed as the main product, and the buffering agent, in the form of the carbonate or the oxide of the cation, which remains behind in the reactor as the ash or the slag from the reaction, would then be recycled back to the fermentation to control pH and repeat the process.

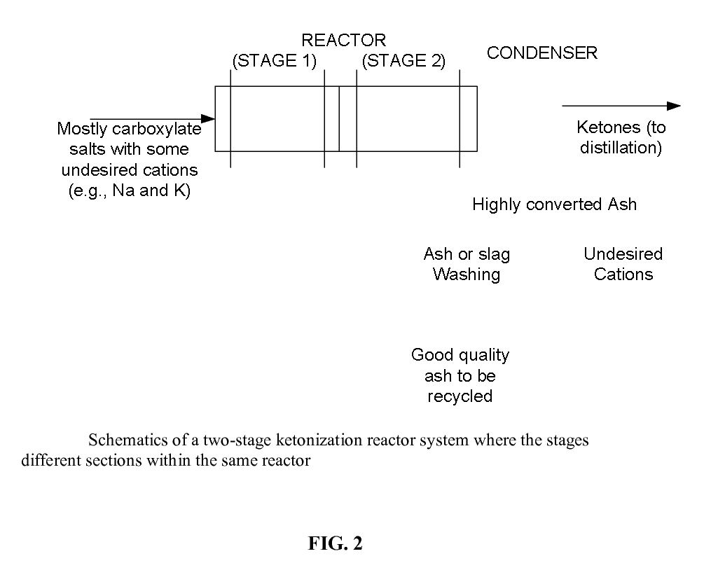

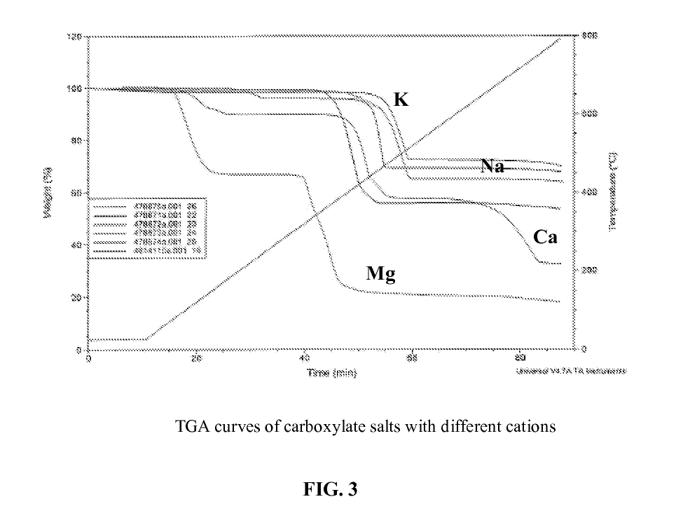

However, with the feedstock used in the fermentation, other cations such as sodium and potassium are also introduced, and these cations, as mentioned, yield less efficient ketonization that requires higher temperatures (i.e., greater than 400° C.) for conversion and that end up with lower yields. In an effort to mitigate the presence of such unwanted cations to maximize yields, a multistage reactor process is proposed. In an embodiment, there may be two or more separate reactors in series. In other embodiments, there may be one big large reactor that may be divided into two or more sections/stages (see FIGS. 2 and 3, respectively). It is noted that while FIGS. 2 and 3 show only two stages, this is only for illustration purposes. Thus, the number of stages is not meant to be limiting, and additional stages are within the scope of the disclosure.

Separating the system into two or more stages, allows implementing different conditions, most importantly temperature, in each reactor or section. Thus, it is possible to start with low-temperature conversion in the first stage (e.g., about 350° C.) where the high concentrations of, for example, magnesium, may allow most of the reaction to occur with little degradation of the end product. Then, any partially converted solids coming out of the first stage may be submitted to higher temperatures (e.g., about 450° C.) in the next stage or to gradually increasing temperatures as it goes from stage to stage if more than two stages are implemented.

It is expected that as the temperature is increased, the degradation of the ketones would increase as well, but having already generated the bulk of the product in the first stages, the degradation would be minimized and the final yields increased. The separation of the reactor system into several stages, especially when several separate reactors in series are used, allow full control of each stage, where not only temperature might be allowed to vary but also other parameters like gas sweep rate, mixing speed, different internals to handle the different solids, etc. The ash exiting the system would be highly converted and it would be sent to a washing step where the highly soluble components would be removed before sending the ash to the fermentation for buffering.

### Example(s)

The following example is of a two-stage ketonization of carboxylate salts generated from a magnesium carbonate/hydroxide buffered fermentation. The feedstock employed in the fermentation is food scraps, which could contain, for instance, sodium chloride from the condiments that are sometimes added to food and other potassium salts. The fermentation broth was concentrated by evaporation to remove as much water as possible. Then a 210 lbs of high-solid concentrate (with 46% solids) was loaded into a high-temperature continuous ketone reactor and the temperature was increased until ˜340° C. was reached. 32.4 lbs of organic phase and 136 lbs of aqueous phase were obtained.

The high-molecular weight ketones that formed were mostly in the organic phase. The aqueous phase contained some of the smaller ketones, such as acetone and 2-butanone, which need to be accounted for to calculate the total yield. The ash solids from the reactor were collected. A small sample was used to determine the amount of unconverted salts still left and thus calculate the conversion. Then 350 g of these salts were loaded into a small reactor for testing the second stage conversion. This time the reaction had to occur at a higher temperature (450° C.). 12.5 g of organic liquid and no aqueous phase was collected. It is important to mention that magnesium salts do not allow very good pH control during concentration through evaporation, as a result, when water is present, free acids are liberated a lost during evaporation with the evaporating water.

In view of this, two yields are reported: the true yield of ketones, and the corrected yield to account for the acid loss as water evaporates. The latter will allow the true efficiency of ketonization reaction to be reflected, and should be the number that is more pertinent to what the embodiments of the disclosure may demonstrate. The measurement of the amount of carboxylate salts in the solids fed and the unreacted ash was done by acidification of these salts and then running the liquid through GC-FID to find the carboxylic acids. The measurement of the amount of ketones produced both in the organic phase and aqueous phase and unreacted acids lost to the aqueous phase were also done using GC-FID. The results for this experimental run are shown in Table 1.

It can be observed from Table 1 that the yields and the final conversion of the salts may be enhanced obtaining an overall conversion of 99% and an overall corrected yield of 90% of theoretical. This is a favorable improvement to one-stage reactions run in the past, where the overall corrected yield was only 80% of theoretical, which was run at >400° C.

TABLE 1
Results from laboratory-scale testing
of two-stage ketonization reaction

| | | % of theoretical | % of theoretical |
|---|---|---|---|
| | Solids | yield** | yield |
| | Conversion* | (True) | (corrected***) |
| First stage | 92% | 82% | 84% |
| Second | 93% | 45% | 45% |
| stage | | | |
| Overall | 99% | 86% | 90% |

*Based on the measured amount of carboxylate salts fed and the measured unreacted salts after the reaction.
**Based on the theoretical yield of ketones to be generated from the measured salts in the feed, and the actual ketones recovered after the reaction both in the aqueous phase and in the organic phase.
***Corrected for the unreacted acid loss to the aqueous phase during the reaction.

### Advantages

Embodiments described herein may provide for an efficient method that mitigates the effect of undesired cations in the thermal conversion of carboxylate salts to ketones. Such mitigation allows the reaction to be run at lower temperatures, while obtaining higher yields and conversions.

A synergistic effect may be realized because methods disclosed herein allow attaining very high yields from the ketonization, and at the same time allows the reaction(s) to occur at lower temperatures. Operating at lower temperatures provides the further advantage of minimizing degradation of desired products.

Further, another advantage of lower temperatures is that they also simplify and decrease the cost of the equipment necessary to run the reaction. For instance, the lower temperatures less than about 350° C. allow the use of conventional heat transfer fluids, unlike temperatures greater than 350° C., which might start requiring molten salts as heat transfer fluids.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations. The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The inclusion or discussion of a reference is not an admission that it is prior art to the present invention, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

What is claimed is:

1. A method for producing organic products, the method comprising:
providing a feed stream comprising a first organic salt and a second organic salt to a reactor;
reacting the feed stream at a first temperature to convert at least some of the first organic salt to the organic products, wherein reacting the feed stream results in a first product stream comprising the organic products and second organic salt;
separating at least a portion of the organic products from the first product stream resulting in a first reduced product stream comprising second organic salt; and
reacting the first reduced product stream at a second temperature to convert the second organic salt to a second organic products.

2. The method of claim 1, wherein the first temperature is different than the second temperature, wherein reacting the feed stream occurs in the reactor, and wherein reacting the first reduced product stream occurs in a second reactor.

3. The method of claim 2, wherein the first organic salt comprises magnesium or calcium.

4. The method of claim 2, wherein the feed stream is resultant from acidogenic fermentation or the alkali treatment of a bioproduct.

5. The method of claim 2, wherein the portion of the organic products and a portion of the second organic products are gaseous.

6. The method of claim 2, wherein the first organic salt comprises an alkali metal.

7. The method of claim 1, wherein the first temperature is less than the second temperature, wherein reacting the feed stream occurs in the reactor, and wherein reacting the first reduced product stream occurs in a second reactor.

8. The method of claim 1, wherein the first organic salt is magnesium organic salt, wherein reacting the feed stream occurs in the reactor, and wherein reacting the first reduced product stream occurs in a second reactor.

9. The method of claim 1, wherein reacting the feed stream occurs in the reactor, and wherein reacting the first reduced product stream occurs in a second reactor.

10. The method of claim 1, wherein the first temperature is less than the second temperature, and wherein reacting the feed stream and reacting the first reduced product stream occurs in the reactor.

11. The method of claim 1, wherein reacting the feed stream and reacting the first reduced product stream occurs in the reactor.

12. The method of claim 1, wherein the first organic salt is produced from a magnesium carbonate or magnesium hydroxide buffered fermentation reaction.

13. A method for improving a pyrolysis reaction, the method comprising:
providing a feed stream comprising an organic salt of certain cations to a reactor;
reacting the feed stream at a first temperature to convert at least some of the organic salt of certain cations to a first organic products, whereby reacting the feed stream results in a first product stream comprising first organic products and unconverted organic salt of certain cations;
separating at least a portion of the first organic products from the first product stream resulting in a first reduced product stream comprising unconverted organic salt cations; and
reacting the first reduced product stream at a second temperature to convert at least some of unconverted organic salt cations to a second organic products.

14. The method of claim 13, wherein the first temperature is different to the second temperature, wherein reacting the feed stream occurs in the reactor, and wherein reacting the first reduced product stream occurs in a second reactor.

15. The method of claim 13, wherein the feed stream is resultant from acidogenic fermentation of a bioproduct selected from the group consisting of agricultural crops, biodegradable wastes, and combinations thereof.

16. The method of claim 15, wherein reacting the feed stream further results in an ash, and wherein at least some of the ash is recycled and used as a buffering agent in the acidogenic fermentation to control pH.

17. The method of claim 16, wherein the first temperature is in the range of about 340° C. to 450° C., wherein the reacting the feed stream is by thermal decomposition, and wherein at least some of the cations of the carboxylate organic salt comprise an alkali metal.

18. The method of claim 13, wherein the first temperature is less than the second temperature, wherein reacting the feed stream occurs in the reactor, and wherein reacting the first reduced product stream occurs in a second reactor.

19. The method of claim 13, wherein reacting the feed stream occurs in the reactor, and wherein reacting the first reduced product stream occurs in a second reactor.

20. The method of claim 13, wherein the first temperature is less than the second temperature, and wherein reacting the feed stream reacting the first reduced product stream occurs in the reactor.

21. The method of claim 18, wherein the feed stream is resultant from acidogenic fermentation or the alkali treatment of a bioproduct selected from the group consisting of agricultural crops and biodegradable wastes.
